(19) United States
(12) Patent Application Publication
(10) Pub. No.: US 2007/0003929 A1
(43) Pub. Date: Jan. 4, 2007

(54) Method for identifying, analyzing and/or cloning nucleic acid isoforms

(76) Inventors: Yoshihide Hayashizaki, IBARAKI (JP); Piero Carninci, Saitama (JP); Alexsander Lezhava, Tokyo (JP); Matthias Harbers, Tokyo (JP); Yuko Shibata, Tokyo (JP)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 10/538,941

(22) PCT Filed: Dec. 12, 2003
(86) PCT No.: PCT/JP03/15956

§ 371 Date: Apr. 28, 2006

(30) Foreign Application Priority Data
Dec. 12, 2002 (JP) 2002-360972

(51) Int. Cl. C12Q 1/68; C40B 30/06; G06F 19/00

(52) U.S. Cl. 435/6; 702/20

(57) ABSTRACT

The present invention provides a method for identifying, analyzing and/or cloning nucleic acid isoforms comprising the steps of: preparing at least two nucleic acid isoforms, complementary to each other, hybridizing the at least two complementary nucleic acid isoforms and forming double strand RNA/RNA or DNA/DNA hybrids comprising unpaired regions; recovering the RNA/RNA or DNA/DNA hybrids comprising unpaired regions from not hybridized nucleic acids and from nucleic acids not comprising unpaired regions; and identifying, analyzing and/or cloning the recovered nucleic acid fragment comprising unpaired regions.

### FIELD OF THE INVENTION

The present invention relates to the identification, sanalysis, selection, preparation and/or cloning of nucleic acid isoforms.

### BACKGROUND ART

The 25-years-old discovery that eukaryotic genes consist of introns and exons was a fundamental breakthrough in our understanding of gene structures and gave rise to a new field in life science focusing on mRNA processing. As introns and exons are both transcribed into a pre-mRNA, an additional step is required to convert the initial pre-mRNA into a mature mRNA, in which the non-coding introns have been removed and the coding exons have been linked together in the correct order. The so-called splicing process, by which introns are excised from pre-mRNAs and exons are re-associated in a specific manner, is essential for the correct processing of mRNA molecules and for a correct translation of the genetic information into proteins.

The pre-mRNA splicing reaction is carried out by splicesomes, which are ribonucleoprotein complexes containing five small nuclear RNAs (snRNAs) and a large number of associated proteins. Splicesomes recognize specific 5′ and 3′ splice sites located at exon-intron boundaries (splice donors and splice acceptors). The following splicing reaction requires that first the 5′ end of the intron is joined to an adenine residue in the branch point sequence upstream of the 3′ splice site to form a branched intermediate, the so-called an intron lariat; in a second step then two exons are ligated and the intron lariat is released from the complex. In this process the exon recognition is a fundamental problem of the pre-mRNA splicing. The splicing machinery must be able to recognize small exon sequences (18 150 bp) located within vast stretches of intronic RNA (on average about 3.5 kb). Moreover, 5′ and 31 splice sites are in general poorly conserved, and introns often contain large numbers of cryptic splice sites similar to a 5′ or 3′ splice-site consensus sequences. Therefore cryptic splice sites can be selected for splicing when normal splice sites are altered by mutagenesis. Beside the splicing donor and acceptor sites, specific sequence elements in exons were characterized as exonic splicing enhancers (ESEs), which interact with a family of conserved serine/arginine-rich splicing factors, the so-called SR proteins. As those ESEs are needed to recruit the splicing machinery and guide it to the flanking 5′ and 3′ splice sites, exon sequences are under multiple evolutionary constraints to conserve not only for the coding information but also for the ability to bind to SR proteins. Such an evolutionary selection may have contributed to the development of mechanisms for stage and tissue specific splicing phenomena.

Once exon recognition is completed, the flanking splice sites of two exons must be joined in the correct 5′-3′ order to prevent exon skipping. Splicing factors, which are bound to the carboxy-terminal domain (CTD) of RNA polymerase II, interact with exons as they emerge from the exit pore of the polymerase. These interactions tether the newly synthesized exon to the CTD until the next exon is synthesized. Although coupling transcription to splicing should prevent exon skipping in constitutively spliced pre-mRNAs, exon skipping can be desired during stage and tissue specific alternative pre-mRNA splicing. In such cases, the presence or absence of regulatory proteins can determine whether or not an exon is recognized and subsequently included in the mature mRNA.

Beside its principle importance for gene regulation and expression, mRNA splicing recently became a focus of genomic research after the sequencing of eukaryotic genomes. The analysis from whole genome sequences from as different organisms as humans, the nematode C. elegans, the fly Drosophila melanogaster, and the complex bacteria Streptomyces reviled unexpectedly small differences in the total number of genes encoded by each genome. Thus the human genome would encode only about 1.5 times as many genes as that of the relatively simple nematode C. elegans. This uncanonical phenomenon may be explained by mechanisms of alternative splicing, which were increasingly applied during the development eukaryotes. Due to differential splicing of its pre-mRNA a single gene can encode for multiple isoforms on the protein level of which each isoform is distinct by alternative exon usage.

An understanding of such alternative splicing mechanisms and the distinct proteins resulting thereof becomes ever more important as an increasing number of reports point to human diseases and aging aberrations related to miss-splicing or a lack of alternatively spliced isoforms. Therefore, there is a huge demand for the detection and characterization alternatively spliced mRNA molecules to allow for the development of novel means in assay development and to identify targets for drug discovery as well as diagnostics.

The identification of sequence variations is thus far a complex and tedious task. In particular for the identification of different splice variants, it is necessary to clone related sequences out of the same or many distinct cDNA libraries and forward individual clones derived thereof to further analysis. Although an initial analysis of individual clones can be performed by restriction digest followed by electrophoretical separation of the resulting fragments, the entire genetic information of the different clones can only be obtained from full-length sequencing and further computational alignments. This process is quite time consuming and cost effective and furthermore does not allow for an up scaling for high throughput analysis. The lack of effective means for the parallel analysis of sequence variations and their application to studies on differentially spliced pre-mRNA molecules is a clear limitation in out of today's genomic research and development projects.

U.S. Pat. No. 6,251,590 discloses a method for identification and/or cloning of differentially spliced nucleic acids from a standard biological sample and a test biological sample. The method consists in preparing a plurality of RNAs from one sample and a plurality of DNAs from the other sample followed by hybridization and formation of hybrids RNA/DNA. The RNA molecule comprising an unpaired region corresponding to the portion of the gene, which is differentially spliced between the samples, is then identified. The method disclosed in U.S. Pat. No. 6,251,590 is limited to the preparation of hybrids RNA/DNA since the strategy for identification of unpaired region is carried out essentially by means of use of enzyme RNase H. This enzyme cuts RNA bound to DNA, but does not cut single strand RNA (the unpaired regione), which can then be recovered. This method however shows several drawback and lack of efficiency. The problem is that 1) the RNase H cuts RNA hybridized to DNA in fragments of 3-10 nucleotides, and 2) RNA which is only partially hybridized DNA is released in the mixture RNA fragments of generally 10-50 nucleotides after cut of RNase H. It results difficulties to distinguish the unpaired region, as it can be a short fragment for example of about 20 bases, from the RNA fragments of 1-10 and 10-50 nucleotides. The researcher needs to carry out a size selection method, for example by electrophoresis, but the presence of impurities cannot be avoided. He therefore, needs to sequence all the recovered fragments in order to determine the fragment corresponding to the unpaired region. This method is therefore not efficient as it results in a high background of false positives and gives rise to artifacts.

The authors of U.S. Pat. No. 6,251,590 propose a further method for recovering of RNA molecule comprising the unpaired region. It consists of carrying out a reverse transcription reaction by using random primers. The problem is, however, that the random primer can hybridize the RNA molecule comprising the unpaired region at any position, including a position inside the unpaired region. The consequence of this strategy is that there is no certainty that the full-length of the unpaired region is recovered. On the contrary, small portions of sequence or fragments of the unpaired region are highly likely to be recovered. Only this strategy therefore results lack of efficiency and accuracy.

There is therefore the need in this field of research of improved and efficient methods, which may assure the identification, selection and preparation of nucleic acids which result from the same or from related genes.

The method of the present invention overcomes the problems of the art and provides and efficient method for the identification, analysis and/or cloning of such nucleic acids.

### SUMMARY OF THE INVENTION

The present invention provides a new, improved and flexible method for the identification, analysis, cloning and/or preparation of nucleic acid variants or isoforms.

The present invention provides a method for identifying, analyzing and/or cloning nucleic acid isoforms comprising the steps of:
a) preparing at least two nucleic acid isoforms, complementary to each other;
b) hybridizing the at least two complementary nucleic acid isoforms and forming double strand RNA/RNA or DNA/DNA hybrids comprising unpaired regions (also indicated as loop);
c) recovering the RNA/RNA or DNA/DNA hybrids comprising unpaired regions from not hybridized nucleic acids and from nucleic acids not comprising unpaired regions; and
d) identifying, analyzing and/or cloning the recovered nucleic acid fragment comprising unpaired regions.

According to a particular aspect of the invention, the recovery step c) as above is carried out by using at least one restriction enzyme which cuts free single strand nucleic acids but does not cut double strand nucleic acids and/or at least one or more restriction enzymes, which cut double strand nucleic acids but does not cut unpaired regions.

The restriction enzymes, which cut double strand nucleic acids but do not cut unpaired regions, can be any kind of restriction enzyme for this purpose. Restriction enzymes, which cut at recognition sites comprising of 4 nucleotides of double strand nucleic acids but do not cut unpaired regions can be used preferable.

According to an embodiment of the inventions, hybrids of DNA/DNA or RNA/RNA comprising unpaired regions are recovered from hybrids nucleic acids not comprising unpaired regions by using nucleic acid single strand-binding molecule, for example single strand nucleic acid-binding protein, antibody, antigen, oligonucleotide, a random oligonucleotide, a chemical group or chemical substance.

The nucleic acid single strand-binding molecule is preferably bound to a tag, for example, biotin, digoxigenin, antibody, antigen, a protein or nucleic acid binding molecule. The tag can be recovered by binding a matrix, for example avidin, streptavidin, digoxigenin-binding molecule, an antibody or its ligand and/or chemical matrix associated with solid matrix surface like metal beads, magnetic beads, inorganic polymer beads, organic polymer beads, glass beads and agarose beads.

According to a further embodiment, the hybrids of DNA/DNA or RNA/RNA can be recovered by using linkers or primers. For example, linkers or primers which recognize specific sequence sites introduced during the preparation of isoforms of step a) as above may be used.

According to a further embodiment, the invention provides a linker system for introducing orientation of sequences. According to one realization, shaped linkers, preferably asymmetric linkers are used to bind the hybrids as above. Preferably, the Y-shaped linkers comprises sticky end, which hybridize to the hybrids or hybrids fragments comprising the unpaired regions. The nucleic acids isoforms oriented with this system can be easily distinguished during sequencing and bioinformatic analysis.

All the hybrids or hybrid fragments comprising the unpaired regions obtained or isolated as above can be stored as such as source of isoforms enriched-libraries or can be analyzed by various means including but not limited to be sequenced and analysis for the determination for genetic information.

Further, the present invention provides a method for using genetic information obtained from the method according to the invention for preparing nucleic acids useful for the subsequent identification, selection, analysis, isolation and/or preparation of further nucleic acid isoforms.

According to one embodiment, the nucleic acids useful for identification and isolation of further isoforms can be applied, fixed and/or printed on a support, like a microarray and used for isoform screening.

According to a further embodiment, the invention provides for computer program or software, preferably applied on a medium, for the prediction, determination and/or analysis of generic information and proteins derived thereof obtained according to the embodiments of the invention.

### BRIEF DESCRIPTION OF THE DRAWINGS

FIG. 1—Principle of mRNA splicing process.

FIG. 2—A general outline of the steps involved in the method of the invention.

FIG. 3—Preparation of strand specific hybridization probes.

FIG. 4—Preparation of PCR products.

FIG. 5—Sample-specific ssDNA synthesis.

FIG. 6—Hybridization of sample-specific ssDNA molecules.

FIG. 7—Incubation of hybridization products with Exonuclease VII.

FIG. 8—Incubation of hybridization products with 4 bp cutters restriction enzymes.

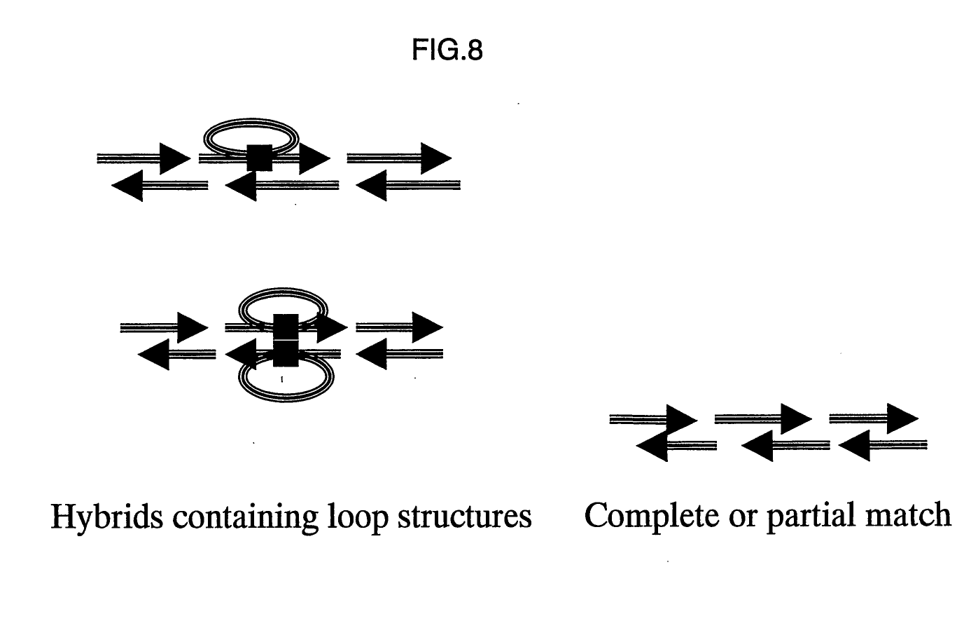

FIG. 9—Capture of loop structures (unpaired region) with biotinylated and randomized oligonucleotide.

FIG. 10—Structure of Y-shape like asymmetric linkers.

FIG. 11—Linker ligation applying Y-shape like asymmetric linkers

FIG. 12—Cloning into vector for sample analysis.

### DETAILED DESCRIPTION OF THE INVENTION

The present invention provides a new, improved and flexible method for the identification, analysis, cloning and/or preparation of nucleic acid variants or isoforms.

For the purpose of the present invention, “nucleic acid isoform” or “nucleic acid variant” means nucleic acids, which differ in sequence and are generated from the same gene or from related genes. In the present description either terms “isoform” or “variant” may be used.

A nucleic acid isoform may be for example but not limited to: 1) the consequence of a mutation, like a deletion and insertation, within a gene; 2) due to alternative splicing of exons and introns within a single primary RNA transcript; 3) be the product of trans-splicing, that is, the splicing of RNA exons generated from both strands of DNA into a single transcript; 4) the product of the same gene at difference stage of development, a different organ or tissue and case of disease and transformation; 5) may refer to nucleic acids generated from related genes; 6) a ‘paralog’, that is, a nucleic acid generated from a gene related to another similar gene by duplication within a genome; 7) a ‘ortholog’, that is, a nucleic acid generated from a gene with similar function to another gene in an evolutionarily related species; 8) a naturally occurring nucleic acid related or similar to an artificial nucleic acid; or 9) an ‘artificial nucleic acid’ related or similar to a naturally occurring nucleic acid”.

The isoforms or variants prepared according to any embodiment of the present invention comprise unpaired regions (or loop) wherein these regions are known, unknown or partially unknown regions.

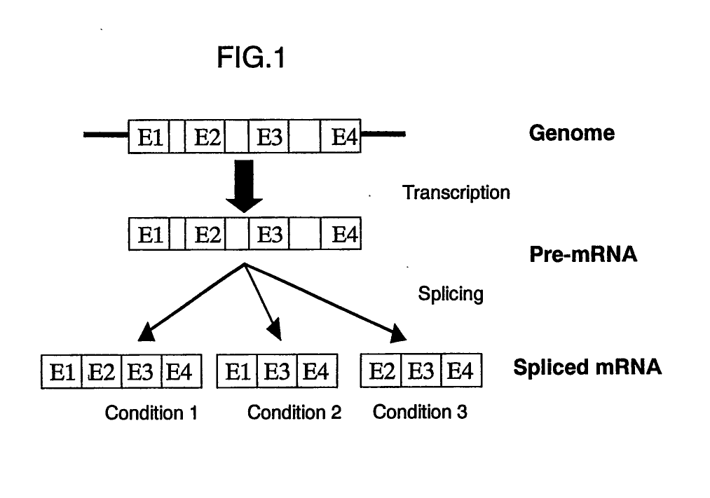

As above said, the unpaired region may be the consequence of different phenomena, including but not limited to alternative splicing process. FIG. 1 shows a schematic example of principle of alternative splicing process.

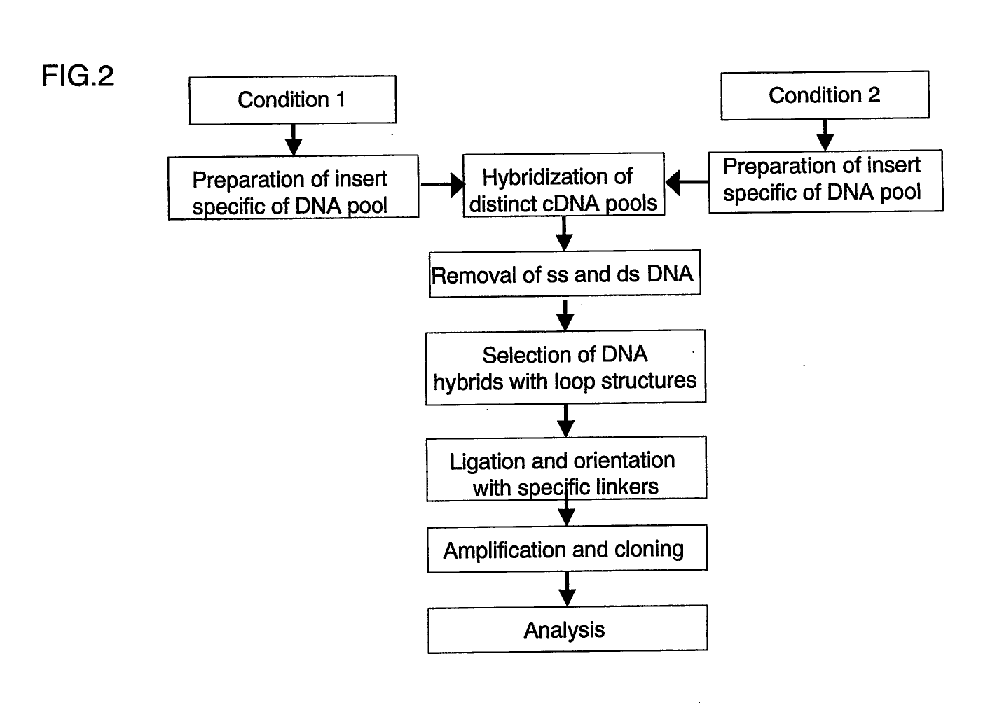

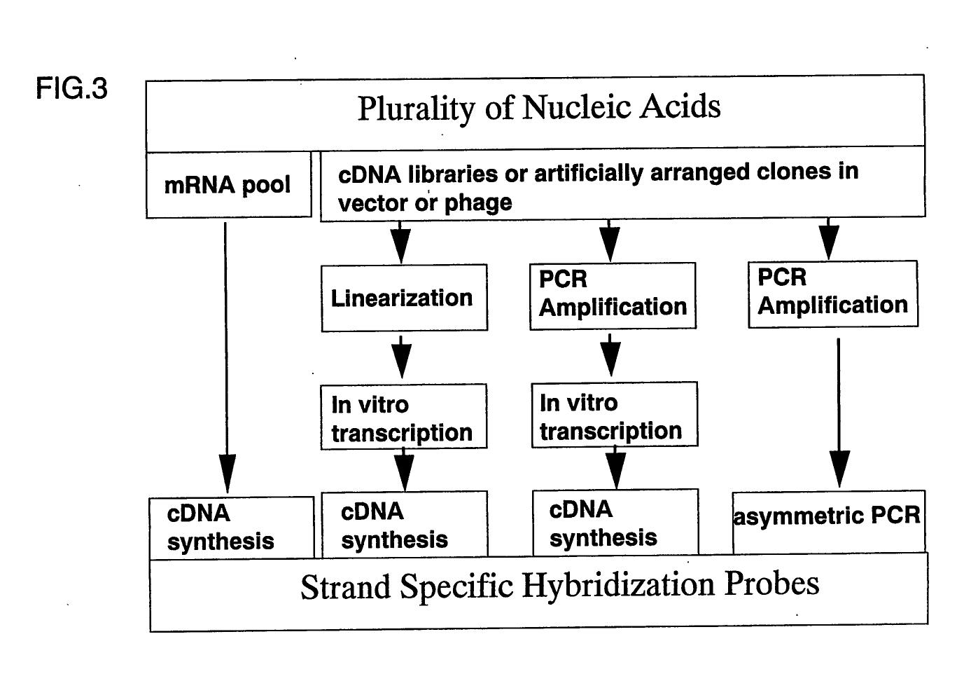

FIGS. 2 and 3, show outlines of the some steps and embodiments of the method according to the invention.

The present invention provides a method for identifying, analyzing and/or cloning nucleic acid isoforms comprising the steps of:
a) preparing at least two nucleic acid isoforms, complementary to each other;
b) hybridizing the at least two complementary nucleic acid isoforms and forming double strand RNA/RNA or DNA/DNA hybrids comprising unpaired regions (also indicated as loop);
c) recovering the RNA/RNA or DNA/DNA hybrids comprising unpaired regions from not hybridized nucleic acids and from nucleic acids not comprising unpaired regions
d) identifying, analyzing and/or cloning the recovered nucleic acid fragment comprising unpaired regions.

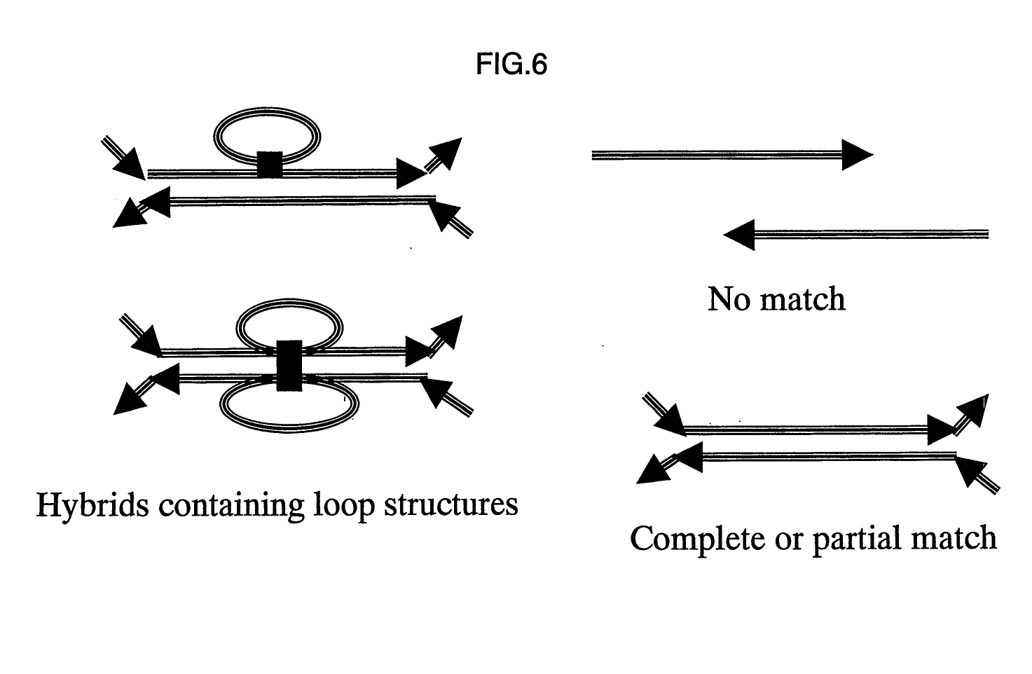

The at least two nucleic acid isoforms have to be complementary to each other, that is, one sense and the other antisense, in order to hybridize and form hybrids of DNA/DNA and RNA/RNA comprising an unpaired region (as shown in FIG. 6).

The at least two nucleic acid isoforms may be obtained from at least one nucleic acid library, biological sample, cell, tissue, organ or biopsy. The isoforms can also be prepared from two or more different nucleic acid libraries, biological samples, cells, tissues, organs or biopsies. The isoforms can be obtained for example from a standard sample and from one or more test sample, as indicated in U.S. Pat. No. 6,251,590 B1, herein incorporated by reference. The test or standard sample can be for example a nucleic acid library, biological sample, cell, tissue, organ or biopsy. The test sample can be preferably a tumoral source, treated cell, and/or from cell undergoing apoptosis or other sources under physiological or pathological conditions as indicated in U.S. Pat. No. 6,252,590.

Samples from different biological stages can also be selected for analysis. These stages can include but are not limited to different time points or developmental stages of the same tissue or cell, or are derived from different tissue samples from the same organism. In another embodiment the invention can be applied to analyze and compare the genetic information of distinct organisms. In its standard application, the invention is used to compare the content of two different samples reflecting on two biologically distinct conditions. However, the invention is not limited to the simultaneous analysis of two samples as mixtures of distinct samples can be applied as well, where depending on the nature of the samples used and their biological context individual samples within a mixture of samples can be distinguished for their origin by specific flanking sequence sites (also indicated as flanking sequence tags or flanking sequence marker sites). In another embodiment of the invention those flanking sequence tags are use to discriminated between samples of distinct origin within a mixture of nucleic acid molecules by differential selection for amplification by specific PCR primers.

As samples individual nucleic acid complementary isoforms prepared from the standard and test samples, nucleic acids or any mixture of individual nucleic acid molecules derived from RNA preparations, from fragments of genomic DNA, or cDNAs can be applied. The invention can use but is not limited to the use of DNA molecules cloned or recombinant into cloning vectors or phages for their better handling and amplification. However, also linear DNA molecules can be directly applied for the invention or made available for the invention by an amplification step. The samples to be compared by the means of the invention can be obtained from any kind of plurality of nucleic acids including but not limited to the use of mRNA, cDNA and genomic DNA samples, and the samples can be mixed and combined in any order depending on experimental needs.

The invention can make use of many kinds of different starting materials and thus the invention is not limited to the use of DNA libraries only. A DNA library can contain any kind of DNA fragment or DNA fragments derived from natural sources or of an artificial nature directly synthesized or obtained by manipulation of genetic material obtained from an organisms, a tissue, a cell line or alike. Furthermore the DNA material cloned into a DNA library can comprise information derived from RNA and transcripted into cDNA or can be derived from fragmented genomic DNA. However, the invention is not limited to the use of nucleic acids derived from a DNA library as any individual DNA fragment derived from natural sources or of an artificial nature directly synthesized or obtained by manipulation of genetic material obtained from an organisms, a tissue, a cell line or alike can be applied to perform the invention and to compare the sequence information of such a DNA fragment to that of one or more DNA fragments. In one embodiment the invention is applied to the analysis of two cDNA libraries, which are compared for their content of nucleic acid isoforms. Then, the isoform complementary molecules can also be prepared from one or more libraries or sample wherein the nucleic acids are subjected to denaturation and re-association.

As standard and/or test sample one or more cDNA libraries can also be used (for instance the cDNA libraries described by Okazaki et al., the Fantom Consortium and RIKEN exploration research group, Nature, December 2002, Vol. 420, 563-573). cDNA molecule can be prepared according to any method known in the art (see Sambrook and Russel, Molecular Cloning, 2001, Cold Spring Harbor Laboratory Press), for example Maruyama K., and Sugano S., 1994, Gene, 138: 171-174 or full-length cDNAs prepared according to the Cap-trapper methodology, which may be normalized and/or subtracted (Carninci et al., October 2000, Genome Research, 10:1617-1630). cDNA library can be prepared inserting cDNAs or full-length cDNA into vectors, for example as described in Carninci et al., September 2001, Genomics, Vol. 77, (1-2):79-90. As indicated above, also genomic DNA, ESTs, RNA and/or mRNA can also be used as starting point for the preparation of complementary nucleic acid isoforms. However, the invention is not limited to the use of two or more pluralities of nucleic acids as one sample can be comprised of a single nucleic acid molecule such as a clone holding a cDNA or a genomic fragment, whose genomic information can be studied by the means of the invention for its presence in a modified or altered or thus alternatively splice variant or variants in any given context of a biological or artificial sample provided in the form of a yet to be different plurality of nucleic acids.

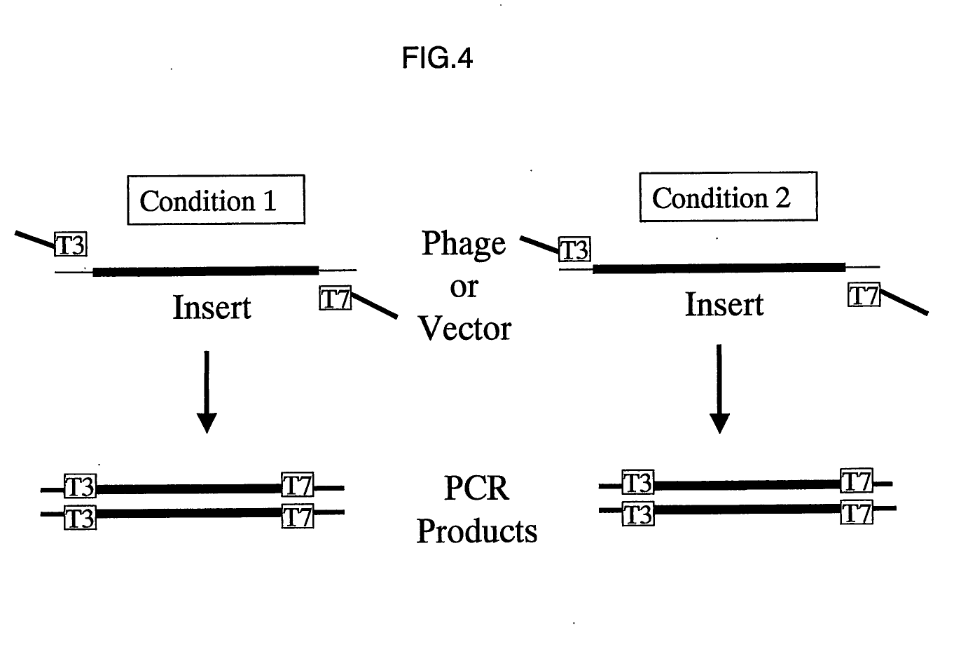

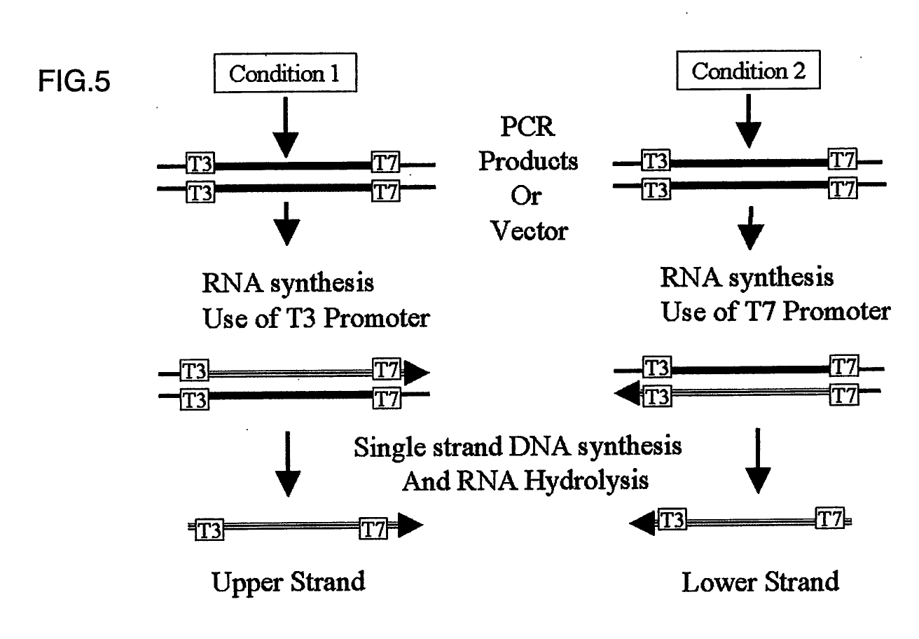

Complementary nucleic acid isoforms (step a) can be prepared to any method known in the art (for example, see Sambrook ans Russel, 2001, as above). According to one embodiment, the complementary cDNA strands are prepared by transcribing sense and antisense isoforms from one or more samples by using at least two complementary nucleic acid isoforms as starting materials by using at least two different RNA and/or DNA polymerases each of them recognizing different promoter sites. According to one realization, RNA transcripts are obtained from the starting materials by using RNA polymerases, which recognize a different promoter site, and cDNAs are prepared from the RNA transcripts by using reverse transcriptase (see FIGS. 4-5). The at least two RNA polymerases recognizing different promoter site are selected from T3 RNA polymerase, T7 RNA polymerase, SP6 RNA polymerase and K11 RNA polymerase or mutant thereof (see U.S. Pat. No. 6,365,350).

Any DNA polymerase for this use known in the art can be used (Sambrook and Russel, 2001, as above). A DNA polymerase and strand specific primers can also be used for this purpose including but not limited to the Taq DNA Polymerase or the DNA Polymerase I Large (Klenow) Fragment, which is Exonuclease minus.

According to one embodiment, as described in FIGS. 2-5, two sets of single stranded DNA molecules are prepared, one set from sample 1 (or condition 1 as indicated in the figures), for example melanocyte full-length cDNA library, and the other set from sample 2 (or condition 2), for example melanoma full-length cDNA library. The two sets of libraries may be amplified, according to standard amplification methodology (see Sambrook and Russel, 2001), for example as phages, as plasmid DNA or as DNA fragments by PCR. The two sets of double strand cDNAs are reverse transcribed using T3 RNA polymerase which recognizes the T3 promoter site in the first set of library and T7 RNA polymerase which recognizes the T7 promoter site in the second set of library, respectively (see FIGS. 4-5). As a consequence, two sets of RNA transcripts, complementary to each other (except for the regions of distinct or missing sequences which are here indicated as unpaired regions or loop) are transcribed. RNA as described and obtained by the means of the invention can be applied to and used directly for performing the invention on the level of RNA/RNA hybrids, whereas in part unpaired and thus loop structure forming hybrids can be enriched by the means of the invention as disclosed below for the isolation of loop structures formed by DNA/DNA hybrids.

Using primers and DNA polymerases, DNA strands are synthesized according to standard technologies (Sambrook and Russel, 2001, as above).

The RNA strands are then removed from the double DNA/RNA strands, by using standard technologies (Sambrook and Russel, 2001, as above), for example causing hydrolysis of RNAs by addition of NaOH.

The products obtained are two sets of single strand DNAs complementary to each other (indicated as lower and upper strands in FIG. 5).

As said above, these two complementary sets of nucleic acids correspond two isoforms, which are complementary to each other except for the regions of distinct or missing sequences, which are indicated as unpaired regions or loop. These unpaired regions correspond, for example, to portions of related genes derived from different loci within the same genome, portions of unrelated genes derived from the same locus within a genome, portions of related genes derived from different genomes. They may correspond to deletions, insertations, exons and/or introns.

The two sets of complementary cDNAs are hybridized in order to form hybrids of DNA/DNA, which comprises one or more unpaired regions, forming structures as shown in schematic form in FIG. 6 (this step corresponds to step b).

The method as above has been described for the preparation of hybrids of DNA/DNA, however hybrids of RNA/RNA are also within the scope of the present invention. Hybrid of RNA/RNA can be prepared according to standard technologies.

Preparation or mixture of hybrids DNA/DNA or RNA/RNA as above described can be stored as source of nucleic acid isoforms-enriched libraries or can used for the isolation of the full-length unpaired region.

The preparation of hybrids of DNA/DNA as above can be treated in order to recovering the hybrids of DNA/DNA comprising the unpaired regions from not hybridized or partially hybridized nucleic acids or nucleic acid regions and from nucleic acids hybrids not comprising unpaired regions.

Both treatments can be carried out independently from each other or simultaneously and in any order.

According to one embodiment, the removal of not hybridized or partially hybridized nucleic acids or nucleic acid regions is carried out by using at least one restriction enzyme which cuts free single strand nucleic acids but does not cut double strand nucleic acids. Restriction enzymes which cut free single strand nucleic acids but does not cut double strand nucleic acids are exonucleases for example: Exo VII, Exonuclease I, Exonuclease T, Lambda Exonuclease, T7 Exonuclease. These kinds of enzymes however are not limited to this list. Further enzymes known to those skilled in this field of the art may also be used. In particular, Exo VII works both in 5′>3′ and 3′>5′ direction, however any other exonucleases working in 3′>5′ direction may be used on their own or in any given combination to reduce the background or artifacts caused by DNA/DNA hybrids with singles stranded DNA overhangs.

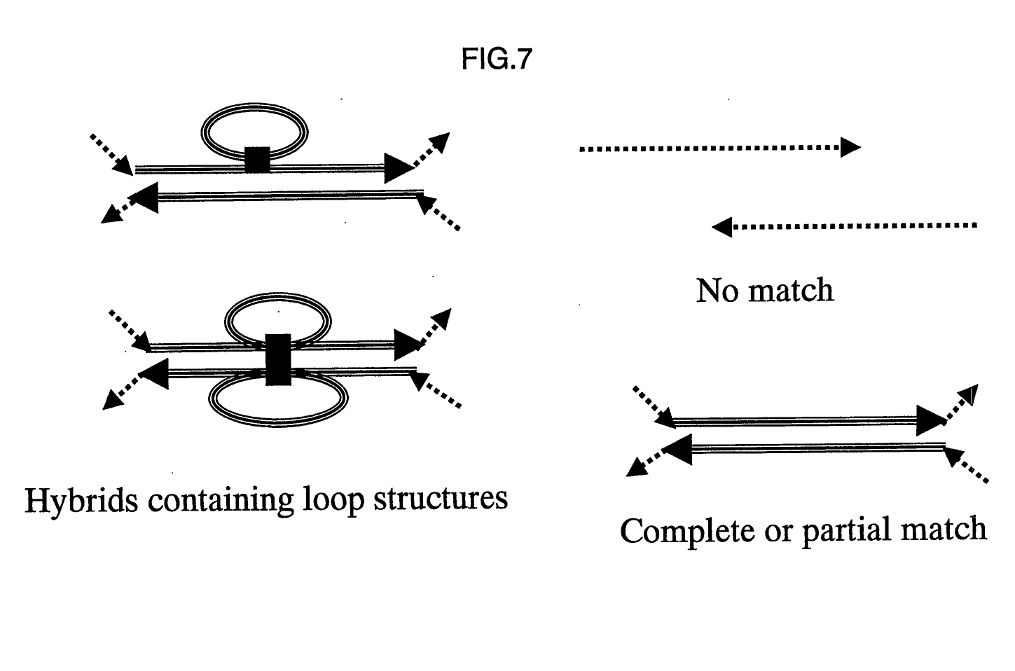

The effect of use of restriction enzymes, which cut free single strand nucleic acids but does not cut double strand nucleic acids, is shown in schematic way in FIG. 7. Single strand nucleic acids, which have not hybridized, and those regions not hybridized are digested by the enzymes as above, leaving only double strand DNA/DNAs.

Following the treatment with Exonucleases as above or independently from that treatment, a step of recovery of DNA/DNA hybrids comprised unpaired regions from hybrids or nucleic acids not comprising unpaired region may be carried out. To this purpose, at least a restriction enzyme, which cut double strand nucleic acids but does not cut unpaired regions, may be used. Preferably, two restriction enzymes, which cut double strand nucleic acids but do not cut unpaired regions are used.

Restriction enzymes, which cut at recognition sites comprising of 4 nucleotides of double strand nucleic acids but do not cut unpaired regions are preferably used. A non-exclusive list of restriction enzymes which cut double strand nucleic acids but does not cut unpaired regions are selected from HapII, HypCH41V, AciI. HhaI, MspI, AluI, BstUI, DpnII, HaeIII, MboI, NlaIII, RsaI, Sau3AI, Taq alpha I and Tsp 509I.

Other suitable restriction enzymes, which are apparent to those skilled in this field of the art may also be used. By using these kinds of restriction enzymes double strands DNA not comprising unpaired regions are cut. Only small fragments of hybrid isoforms comprising the unpaired regions are not cut by these enzymes.

The method of treatment for removal of unpaired regions from not hybridized nucleic acids and from nucleic acids not comprising unpaired regions by using at least one restriction enzyme which cuts free single strand nucleic acids but does not cut double strand nucleic acids (as above disclosed) and/or at least a restriction enzyme which cut double strand nucleic acids but does not cut unpaired regions (as above disclosed) are useful for the method according to the invention (as outlined in FIGS. 2 and 3) however are not limited to that use. Accordingly, a general method for the recovery and isolation of nucleic acids comprising one or more unpaired regions by using either or both the above methods of use of restriction enzymes is also within the scope of the present invention.

Preparation of hybrid isoforms comprising the unpaired regions can be recovered and isolated according to methodologies known in the art. For example by using single strand nucleic acid-binding molecule. Single strand nucleic acid-binding molecule may be a single strand nucleic acid-binding protein, antibody, antigen, oligonucleotide, a chemical group or chemical substance. The oligonucleotide can be an oligonucleotide having random sequence, preferably a random oligonucleotide of 15-30 nucleotides, preferably of 25 nucleotides (it may be indicated as “25N”). A single strand nucleic acid-binding protein can be any protein having this characteristic (see Sambrook and Russel, 2001, as above). Proteins capable of binding single strand nucleic acids can be for example the E. coli single-stranded DNA binding proteins (SSB) produced by Promega, Catalog number M3011, which bind with high affinity to single-stranded DNA but do not bind to double-stranded DNA (see also Sancar et al., 1981, Proc. Natl. Acad. Sci., USA 78, 4274; Krauss et al., 1981, Biochemistry, 20, 5346). Single strand nucleic-binding proteins are also disclosed for example in EP 1041160 A1 (incorporated by reference). Other single strand nucleic acid-binding substances are disclosed for example in EP 0622457 A1 (incorporated by reference).

Single strand nucleic acid-binding substances are preferably bound to a tag molecule. A tag molecule may be selected form biotin, digoxigenin, antibody, antigen, a protein and nucleic acid binding molecule. The single strand nucleic acid-binding molecule/tag molecule complex may be recovered by using a matrix. A matrix may be selected from avidin, streptavidin, digoxigenin-binding molecule, an antibody and its ligand and/or chemical matrix. The above lists of tags and matrices are, however, not limited to the compounds above indicated.

When the tag is biotin, the matrix may be avidin or streptavidin. When the tag is digoxigenin, the matrix may be digoxigenin-binding molecule (see Roche Catalog). When the tag is an antigen, the matrix may be the antibody. The single strand nucleic acid-binding molecule can also be covalently attached to the matrix. For example is case of oligonucleotide with an amino group, which can be used for covalent binding.

The recovery of the desired nucleic acid isoforms is preferably carried out when the matrix is conveniently associated to a solid matrix surface. The matrix solid surface may be selected from metal beads, magnetic beads, inorganic polymer beads, organic polymer beads, glass beads and agarose beads. Inorganic polymers include silica, ceramics, and the like organic polymers include polystyrene, polypropylene, polyvinyl alcohol, and the like. Metals include iron, copper, and the like.

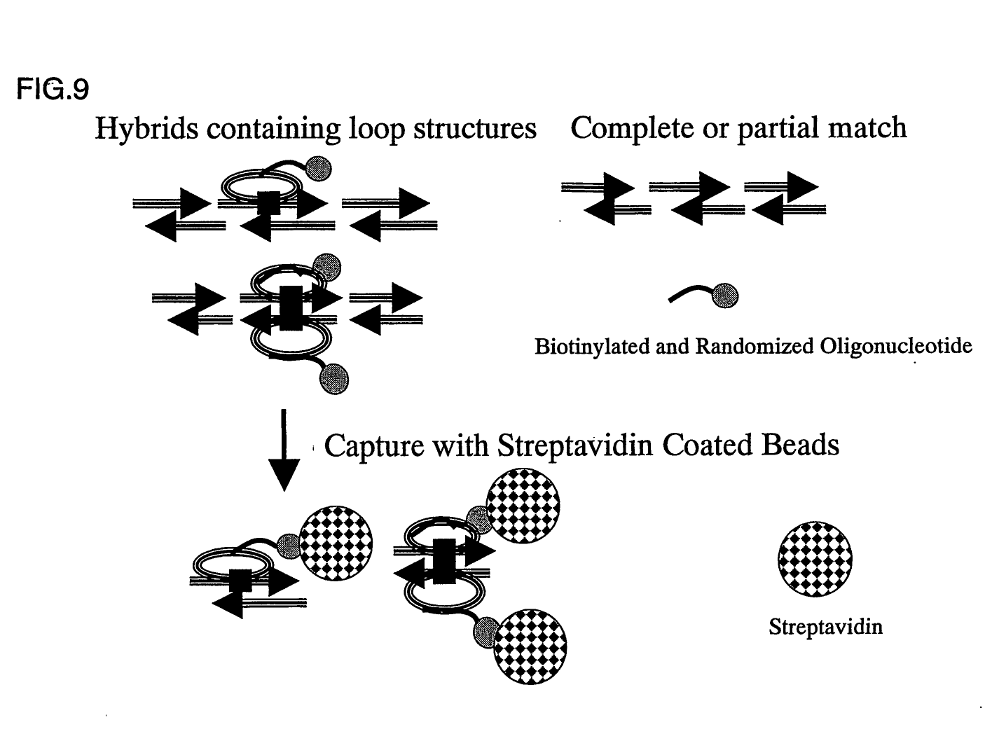

Examples of tags, matrices and matrix solid surfaces can be found in EP 0622457 A1 (incorporated by reference). A schematic example of the recovery as described above is shown in FIG. 9.

Hybrids of DNA/DNA or RNA/RNA isoforms comprising unpaired regions are isolated in this way from hybrids not comprising unpaired regions and are recovered by being released from the single strand nucleic acid-binding molecule according to standard methodologies, for instance by heating, for example 40-60, preferably 50 degrees C. In case of use of random oligonucleotide as single strand nucleic acid-binding molecule, a light heat is enough for releasing the hybrid isoforms from the single strand nucleic acid-binding molecule because the random oligonucleotide is not perfectly hybridized.

Preparations of isoforms as obtained from the above method can be stored as isoforms-enriched libraries or can be processed for the next step for the preparation of isoform with unpaired regions.

One situation which may happen in preparing hybrids of DNA/DNA and RNA/RNA is that the two DNAs (or the two RNAs) of the hybrid lack orientation, and during sequencing and/or further bioinformatic analysis is not clear if the two DNAs (or two RNAs) are complementary or sense molecules.

In order to overcome this problem, present inventors also provide a method for introducing orientation into each strand of the hybrid isoforms and this method represent a further embodiment of the present invention.

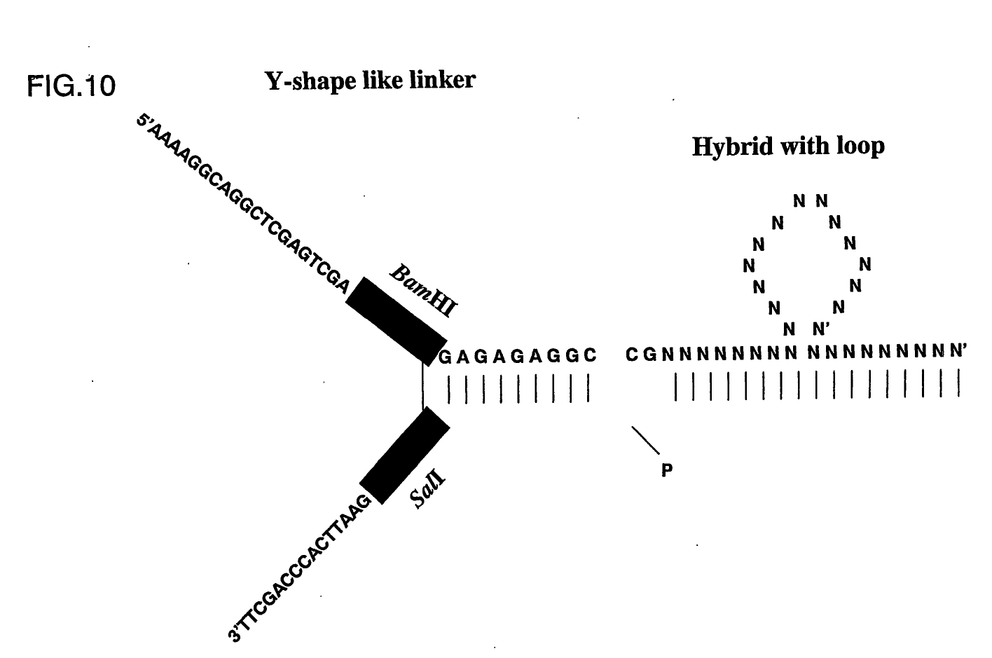

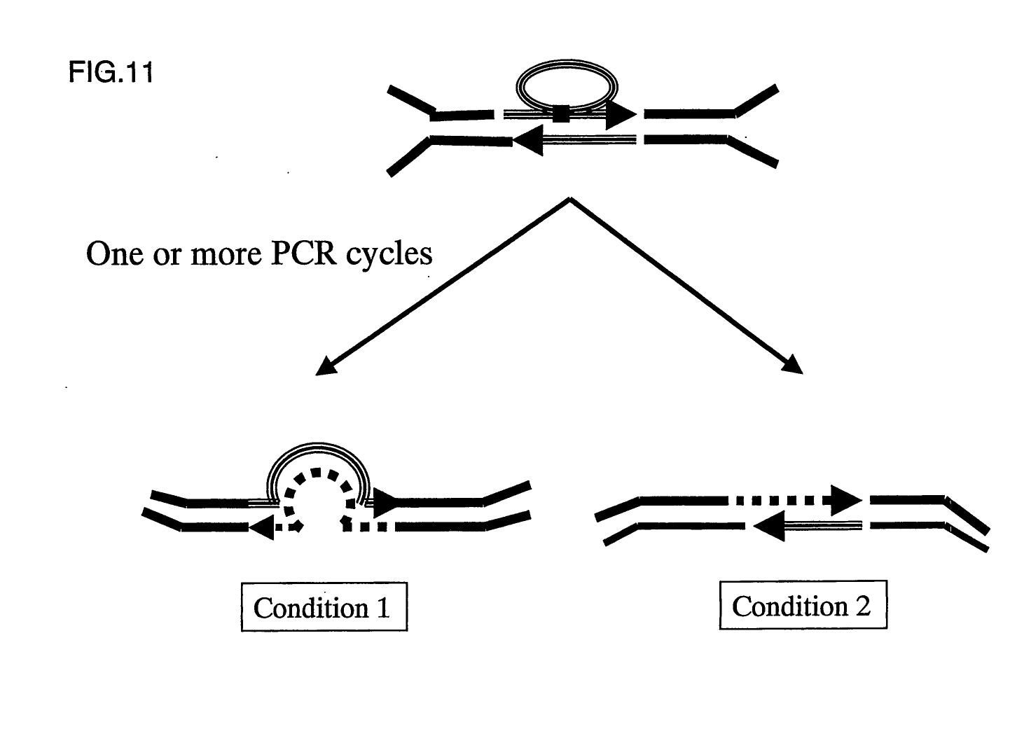

This embodiment consists in the preparation of Y-shaped linkers (see FIGS. 10 and 11). These kinds of linkers consist of a double stranded body region and two single strand arms. Y-shaped linkers have been disclosed for example by Tazavoie and Church, 1998, Nat. Biotechnology, 16: 566-571.

According to the embodiment of the invention, each arm of the Y-shaped linker comprises a different specific marker site sequence or tag sequence. For instance, one arm may have the marker sequence (1) and the other arm the marker (2). When sequenced and analysed the sequences having the marker (1) and the nucleic acid sequences having the marker (2) will be treated as complementary nucleic acid sequences. One or more kind of Y-shaped linkers can be used at the same time if required to provide distinct overhangs for ligation. However, beside the overhang for the ligation only one kind of linker can be used (see also FIGS. 10, 11).

The Y-shaped linker can be attached to the hybrid DNA/DNA or RNA/RNA isoforms recovered according to any method known in the art (Sambrook and Russel, 2001, as above). For example by using RNA or DNA ligase. Examples of these ligases are T4 DNA ligase, E. coli DNA ligase, RNA ligase, T4 RNA ligase.

According to a preferred embodiment, the Y-shaped linkers have a sticky end, at the end of the double stranded body, which hybridizes to the sticky ends of the hybrid double strand nucleic acids to be recovered (in the present case hybrid DNA/DNA or RNA/RNA isoforms comprising the unpaired region). Specific sticky ends of the hybrid nucleic acids can be introduced by specific restriction enzymes. For example, when 4 cutter restriction enzymes, as above indicated, are used to digest double stranded nucleic acids, the Y-shaped linkers can be prepared having sticky end capable to hybridize to the hybrid DNA/DNA isoforms sticky ends.

According to another embodiment, the sticky end of the linker are of random sequence so that they can hybridize to any kind of sticky end of the hybrid nucleic acids.

The use of the Y-shaped linker to impart orientation is not limited to bind the hybrid double stranded isoforms of the invention but can applied in general to recover and to impart orientation to any double stranded nucleic acids. Accordingly, the present invention also discloses a method for imparting orientation to the two strands of double stranded nucleic acids by using Y-shaped linkers as above described.

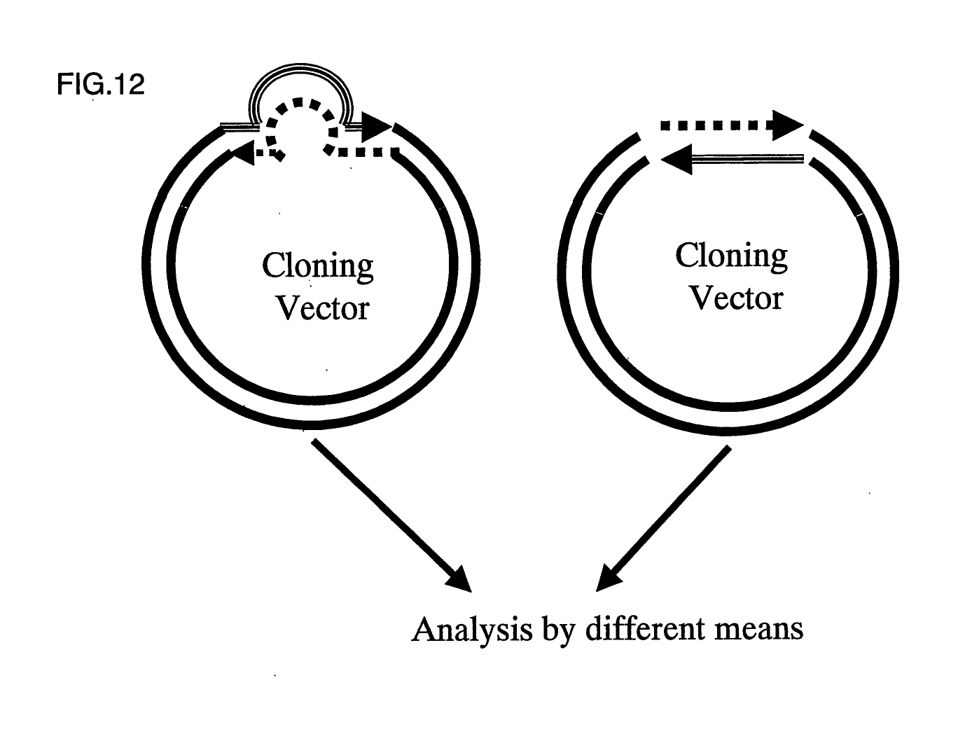

The hybrid DNA/DNA or RNA/RNA isoforms comprising the unpaired region bound to the linkers disclosed as above can be amplified, for instance by one or more cycles of PCR (see FIG. 11) and cloned (see FIGS. 11, 12). The cloning can be carried out according to any technique known in the art (see for example Sambrook and Russel, 2001, as above). For example using cloning vectors (see FIG. 12). Methods for preparing cloning vector and cloning is disclosed for example in WO 02/070720 A1 (incorporated by reference).

With reference to systems for recovering and/or cloning hybrid DNA/DNA or RNA/RNA isoforms comprising the unpaired regions, other methods are available. For example hybrids of RNA/RNA can be recovered and/or cloned by reverse transcription upon the RNA/RNA hybrids, according to standard methods, by using primers which recognized specific sequence sites (also indicated as recognition sites or sequence tags) of the RNAs which may have been introduced in the library phage or vector, during amplification step (FIGS. 3, 4), or during the synthesis of RNA (FIG. 5). For instance, with reference to FIG. 4, the specific recognition sites can be introduced with the primers comprising the T3 and T7 promoter sites.

The isoform as recovered as above in a cloned vector (FIG. 12) can be introduced intro a host cell according to standard methods (Sambrook and Russel, 2001, as above). The present invention therefore also provides for a method for the preparation of polypeptides comprising culturing the host cells as above.

Polypeptide of recovered isoform nucleic acids of the invention can also be prepared according to other known techniques like using cell-free in vitro (Kigawa et al., 1999, FEBS Lett., 442, 15-19 or in in vivo systems.

The isoforms comprising the unpaired regions included in cloning vector can be sequenced and analysed.

The invention provides means for the preparation of DNA libraries specifically enriched for sequence isoforms, which define the difference between of two or more pluralities of the nucleic acid molecules. The libraries obtained according to the invention can be analysed by and applied to standard techniques known to a person skilled in the state of the art of molecular biology (see for example Sambrook and Russel, 2001, as above). The sequencing can be also carried out according to the description in Shibata et al., November 2000, Genome Research, Vol. 10, (11): 1757-1771. This applications include but are not limited to partial or full-length sequencing of the insert, the preparation of probes for hybridization experiments, and the sub-cloning or recombination of the inserts or parts thereof into other DNA molecules to allow for their manipulation or expression in the form of RNA or proteins. The recovered isoform comprised into the cloning vector can be in fact transferred into a vector suitable for sequencing, for instance according to the method described in Carninci et al., September 2001, Vol. 77, (1-2), 79-90.

In another embodiment, the invention provides means for the analysis of sequence information derived from DNA or RNA molecules obtained during the realization of the invention. As those selected DNA or RNA molecules are enriched for DNA or RNA isoform fragments, which are distinct between the two or more analyzed samples, the sequence information derived thereof is a valuable source of information to analyze the use of genetic information during different biological stages. The analysis of sequence information is initiated by multiple alignments of the DNA sequences against one another to reduce the redundancy in the sequence set derived from one experiment and for the grouping of sequences with the same orientation marker. Sequences with the identical orientation markers are derived from the same input sample or mixture of samples. The distinction between at least two orientation markers allows tracking back the origin of each sequence and related clones in the cause of the invention. Due to the experimental approach of the invention each sequence should contain information on the flanking region as well as the sequence variation. Thus the invention allows for the identification of boarders of the sequence variants and the identification of the neighboring regions in the initial nucleic acid samples. Individual sequence information can be further analyzed by searches in reference databases known to a person skilled in this field of the art. Any methodology, for example bioinformatic method, for alignment and obtainment of information can be used. Information obtained from alternative spliced nucleic acids can be analysed by means of bioinformatic approaches, for example by aligning the alternative sliced information to genomic sequence data by using computational tools (TAP) in order to discover their function. The understanding of the function of alternative spliced molecules is very valuable in research since alternative splicing is implicated in human diseases (Kan et al., 2002, Genome Research, 1837-1845). Searches in reference database could include but are not limited to alignments to partial and full-length cDNA as well as genomic DNA sequences. The initial sequence information may be extended by alignments to reference sequences, which may allow for a more throughout sequence analysis on the use of the genetic information and proteins derived thereof. In yet another embodiment the invention can be used and applied for the identification and analysis of introns and exons within transcripted regions of the genome and their selective use within spliced mRNA molecules. Here the invention can provide also relevant information on the coding regions of differentially spliced pre-mRNA molecules and the proteins derived thereof. In yet another embodiment the invention provides intron or exon specific nucleic acid molecules for further manipulation or as experimental tools for the cloning and characterization of differentially spliced mRNAs.

The invention provides effective means for the analysis of sequence variations by matching two or more pluralities of nucleic acids. Out of the selective enrichment of DNA hybrids consisting of loop structures plus double-stranded flanking regions and assembled out of two DNA strands with distinct orientations to mark their origin, the invention allows for the isolation and characterization of those sequence variations comprising and indicating the differential use of related genetic information between the samples. Thus the invention provides novel means for the analysis of differentially spliced pre-mRNA molecules in any biological context. Due to the universal layout of the approach, the invention permits for but is not limited to the analysis of highly complex nucleic acid mixtures by comparing entire pools of mRNA or cDNA molecules derived from mRNA preparations or cDNA libraries. The invention can also be applied in a more focused manner where only different splice variants of the same pre-mRNA or a given transcripted region in the genome are investigated. By applying the invention nucleic acid molecules and sequence information derived thereof can be obtained for further analysis to allow for the functional characterization of known nucleic acids or the identification and isolation of thus far unknown nuclei acids. As the invention can be employed in a wide range of applications in gene discovery and genomic research the approach will greatly contribute to academic and commercial research and development in the field.

Accordingly, the invention provides for a method for identification of isoform nucleic acids and/or polypeptides by using the information obtained by the analysis of the isoform sequences recovered according to any embodiment of the invention.

The invention also provides for a method for the detection and/or isolation of nucleic acid isoforms comprising the steps of:
i) preparing at least one oligonucleotide probe comprising the whole or part of sequence of an unpaired region identified and/or cloned according to any embodiment of the invention; and
j) hybridizing the oligonucleotide probe to nucleic acids comprising nucleic acid isoforms;
k) isolating the nucleic acid isoforms.

The oligonucleotide probe prepared as above can be used to isolate full-length nucleic acid isoform. The oligonucleotide probe may comprise at least one exon or intron. The nucleic acid probe can also be prepared using chemical synthesis methods known in the art using the sequencing and bioinformatics information obtained according to the invention.

The present invention also disclose a nucleic acid probe obtained as above described.

The determination of sequence variation of isoforms prepared according to any embodiment of the invention may comprise the full-length or partial sequencing of the isoform.

According to a further embodiment, the sequence information of the sequence isoforms is used for the design of sequencing primers. The invention therefore also provides for such primers designed with a sequence suitable for sequencing.

The sequencing data of the isoforms obtained by any embodiment according to the invention can be analysed are alignment to the genome, to genomic sequencing data and/or to cDNA sequencing data to obtain genetic information. The information so obtained may be information of alternative splicing.

The invention further relates to the use of the information, obtained from the sequencing and/or analysis method according to the invention, for the detection and/or diagnosis of a disease, disease condition, pathology, a physiological condition, for assessing toxicity, for assessing the therapeutic potential of a test compound and/or for assessing the responsiveness of a patient to a test or treatment. Example of use for this kind of detection, identification and/or diagnosis of disease or physiological and/or pathological condition has been described in U.S. Pat. No. 6,251,590 B1 (incorporated by reference).

The invention furthermore relates to the use of isoforms obtained according to any embodiment of the invention and/or to the nucleic acid probe prepared as above for the preparation of non-soluble supports for hybridization in situ. Accordingly, the invention refers to a non soluble support comprising at least a nucleic acid comprising an unpaired region prepared according to any method of the invention, a nucleic acid complementary to the unpaired region and/or the probe prepared as above, fixed, applied and/or printed thereon.

An example of support having nucleic acid or polypeptide molecules is described in U.S. Pat. No. 6,258,542 B1 (incorporated by reference) for storing and/or delivery.

Other non solid support, preferably on solid matrix, comprising comprising at least an nucleic acid comprising an unpaired region prepared according to any method of the invention, a nucleic acid complementary to the unpaired region and/or the probe prepared as above, fixed, applied and/or printed thereon can be used for hybridization in situ. An example of this support is biochip and/or microarray. Accordingly, any microarray comprising any isoform, unpaired region and/or probe according to the invention is within the scope of the invention. Microarray can be prepared and used according to standard technologies, for example as described in Sambrook and Russel, Molecular Cloning 2002, Cold Spring Harbor Laboratory Press.

Microarray prepared in this way can be used for the identification and isolation of further known or unknown nucleic acid isoforms.

The support or microarray prepared according to the invention can be used for the detection and/or diagnosis of a disease, disease condition, pathology, a physiological condition, for assessing toxicity, for assessing the therapeutic potential of a test compound, for assessing the responsiveness of a patient to a test or treatment, for the detection of nucleic acids and/or for the detection of nucleic acid isoforms. Accordingly, the invention relates to the use of genetic information obtained according to any embodiment of the invention for detecting and/or isolating nucleic acids from a support, microarray, nucleic acid library, biological sample, cell, tissue, organ and/or biopsy.

According to a further embodiment, the invention relates to a computer program and/or software applied on a medium for the analysis of genetic information obtained according to the sequencing and analysis of information as above described. The computer program and/or software applied on a medium can be used for the alignment of the nucleic acid isoforms sequences or information obtained according to any embodiment of the invention to genomic and/or cDNA sequence information.

The computer program and/or software can also be used for the prediction, determination and/or analysis of functional domains of polypeptides that derive from nucleic acid isoforms sequence or information obtained according to any embodiment of the invention.

The sequence analysis according to the invention will one or more of the following elements, features, steps and/or considerations:
QC on sequencing reads and definition of cutoffs for “useful reads”;
grouping of sequences depending on orientation marker to indicate their origin;
alignment of reads to group them into clusters to reduce the redundancy in the set for further analysis and statistical analysis of clusters;
alignment of representative clusters to public or preparatory data sets and analysis of the results;
mapping of representative clusters to genomic information where possible;
analysis of genomic regions based on the mapping results, information available on the locus including but not limited to predicted or identified intro-exon structures;
confirmation of already identified, predicted or newly recognized exons or introns;
design of computational means to confirm splice sites and to rank them according to their reliability; filtering out artifacts;
computational means for prediction of or translation into proteins encoded by or modified by exons identified due the cause of the invention;
design of specific probes for the analysis of exons or introns by hybridization;
design of specific primers for the analysis of exons or introns by PCR;
listing of recognition sites for restriction enzymes;
design of specific primers for the analysis of exons and introns by sequencing reactions.

The present invention further relates to analysis of nucleic acids obtained by any embodiment of the invention. These nucleic acids may be used for the design and preparation of support, in particular macro- and micro-array. The nucleic acids obtained by amplification, for example PCR, may be analyzed by any embodiment of the invention. The nucleic acids obtained by any embodiment of the invention by amplification, for example PCR may be analyzed, followed by analysis with a set of restriction enzymes. The nucleic acids obtained by any embodiment of the invention may be analyzed by partial or extended sequencing using specific sequencing primers. The nucleic acids obtained according to any embodiment of the invention may be used for the cloning of cDNA, of a genomic DNA and/or for chemical synthesis of a DNA or RNA molecule. The nucleic acids obtained according to any embodiment of the invention may be used for the synthesis of a protein partially or entirely encoded by the nucleic acid. The comparison of nucleic acids obtained by any embodiment of the invention derived from two or more different biological samples may be applied. The comparison of nucleic acids obtained by any embodiment of the invention derived from a cDNA or from a fragment of genomic DNA to samples derived from one or more different biological samples may be applied.

The present invention will be further explained in more detail with reference to the following examples.

### EXAMPLE

### Example 1

Protocol of Alternative Splicing Exon Library Method

Full-length cDNA libraries from cell line cultures of melanocyte and melanoma were constructed using the method developed by Carninci et al. Genome Res. 2000 October;10 (10); Carninci et al. Genomics. 2001 September;77 (1-2):79-90. We can use other method by developed by Maruyama, K., Sugano, S., 1994. Gene 138, 171-174. Lambda vector pFLCII (Derivative of the ampicillin-resistant plasmid pBlueScriptII-SK(+), Carninci et al., 2001, Genome Research, Vol. 77, (1-2), 79-90). cDNA sequences were inserted into vector with the XhoI site at the 5′ end of the cDNA and the BamHI site at the 3′ end.

We sequenced the 5′ ESTs using the T7 primer and the 3′ ESTs using the T3 primer. The following can be used for the library construction. Stock of the library-phage solution was made by adding 70 ml of DMSO (Dimethyl Sulfoxide, Wako Chemical, Japan) to 930 ml of phage solution and mixed gently manually. The stock was kept at −80 degree C.

Part 1. DNA Extraction from Amplified Phage.

1 ml of phage stock solution was mixed gently with addition of RNAse, 10 u/μl and DNAse, 1 u/μl (both Promega), 2 μl of each enzyme, respectively. Solution was incubated on 37 degree C. for 20 min. After that, 500 μl of pre-swollen microgranular anion exchanger DE52 (Diethylamoioethyl cellulose, Whatman) was applied with keeping manual mixing for about 10 min. Mixture was centrifuged for 1 min at room temperature using 10,000 rpm. Supernatant was transferred to a 1.5 ml new tube and was centrifuged with the same condition as above. After the second centrifugation supernatant was transferred to 2 ml new tube and was incubated on 37 degree C. for 5 min with 100 μl of 1M ZnCl2. After centrifugation white pellet was visible and supernatant was discarded. Pellet was well re-suspended with 100 μl 0.5M EDTA and 900 μl 7M of Gu-HCl and 100 μl of matrix (Diatomaceous Earth, Sigma) were applied. After gentle and well mixing for about 5 min., solution was centrifuged, 80011 of upper phase was discarded and the remaining part (about 400 μl) was applied to the filter unit (Empty Micro Bio-Spin column BIORAD), placed into 1.5 ml tube. Solution was spin down by brief centrifugation for 1 min at 12,000 rpm at room temperature and flow through was discarded. Filter was washed with 400 μl 7M Gu-HCl, wash solution (twice) and 80% ETOH (twice) with 400 μl for each time. Filter unit was transferred into 1.5 ml tube and 100 μl of pre-warmed TE was applied in the middle of filter. After 2 min. it was centrifuged and 5 μl of DNA solution was applied to the agarose gel (NuSieve GTG Agarose, TAKARA) (according to Sambrook and Russel, 2001, as above) for concentration and quality checking. DNA solution was further purified using S400 column (Amersham Pharmacia). Sample was applied and flowed trough the column using centrifuge on 3000 rpm for 1 min. at 4 degree C.

PCR Amplification of Inserts.

DNA solution has been used further for PCR amplification of inserts. PCR primers were designed for the vector pFLCII (Carninci et al., September 2001, Vol. 77, (1-2), 79-90) part with possible close approach to the sequences of inserts. Phage promoter sequences T3 and T7 were attached to the PCR primers and incorporated to both the PCR products. Reaction conditions were as follows: 2.5 μl of each 10 μM of primer:

| | |
|---|---|
| T3GW1: | |
| GAGAGAGAGAATTAACCCTCACTAAAGGGACAAGTTT | (SEQ ID NO:1) |
| GTACAAAAAAGC | |
| and | |
| T7GW2: | |
| GAGAGAGAGAATTAACCTCACTAAGGGACCACTTTGT | (SEQ ID NO:2) |
| ACAAGAAAGC. | |

Template 4 μl (about 40 ng), 2×GC buffer 50 μl mM dNTPs 16 μl, H₂0 25 μl. Hot start at 95 degree C., add 1 μl LA Taq (all TAKARA, Japan). PCR was performed using 10-20 cycles: 95 degree C. for 1 min. 55 degree C. for 30 sec. and 72 degree C. for 8 min. After reaction, proteinase K digestion was conducted followed by extraction with phenol/chloroform and chloroform (Carninci and Hayashizaki, Methods Enzymol. 1999; 303:19-44), and cDNA was precipitated and dissolved in 100 μl of H₂0.

RNA Synthesis.

RNA was synthesized was carried out by using T3 RNA polymerase (Life Technologies, BRL, 50 u/μl), to prepare sense run-off RNAs. T7 RNA polymerase (Life Technologies, BRL, 50 u/μl) was used to prepare antisense run-off RNAs, 10 μl of PCR sample (3 μg) has been used as a template and reaction mixture was incubated for 5 hrs. at 37 degrees C. Reaction was performed using the following condition: 3 μl of T7 or T3 RNA polymerase was added 40 μl of 5×T7/T3 buffer (Life Technologies, BRL), 20 μl of 0.1M DTT (Life Technologies, BRL), 1.6 μl of 10 mg/ml BSA (Life Technologies, BRL), 10 μl of 10 mM rNTP (Boehringer Mannheim), 115.4 μl of H₂O with total volume of 200 μl.

Solution gradually turned to the white and RNA was synthesized. After that, DNAseI (RQ1, RNase-free, Promega, 1 u/μl) treatment was performed for about 30 min: With addition of 20 μl of 10 mM CaCl₂ and 1 μl of DNAse. Sample was dissolved with 100 μl of water and further purification with QIAGEN purification Kit(QIAGEN) was employed in accordance with the manufacturer's instructions. Final volume of solution was adjusted in 100 μl of water. Then, proteinase K digestion was conducted followed by extraction with phenol/chloroform and chloroform, and cDNA was precipitated.

1st Strand cDNA Preparation.

A solution of 5 μg of RNA sense strand (31 μl) were combined to 5 μl of first-strand primer (SEQ ID NO:2) for a total volume of 36 μl (solution A). 5 μg of RNA antisense strand (31 μl) were combined to 5 μl of the other first-strand primer (SEQ ID NO:1) for a total volume of 36 μl (solution B). Each of the two solutions (sol A) and (sol B), independently, was denatured at 65 degrees C. for 10 min and put in two tubes (one containing denatured sol A and the other containing denatured sol B). Simultaneously, 100 μl of 2× of buffer GC (TAKARA), 20 μl of 2.5 mM dNTPs, 40 μl of saturated trehalose (approximately 80%, low metal content; Fluka Biochemika), and 4 μl of Superscript. II reverse transcriptase (Invitrogen) (200 u/μl) were combined to a final volume of 164 μl (solution C). Further, 0.2 μl of [32P]dGTP were placed in a third tube. Solution A was mixed on ice with solution C, and an aliquot (20%) of the mixture was quickly added to the tube containing the [32P]dGTP. First-strand cDNA synthesis was performed in a thermocycler with a heated lid (MJ Research) according to the following program: step 1) 45.degree C. for 2 min; step 2) gradient annealing: cooling to 35.degree C. over 1 min; step 3) complete annealing: 35.degree C. for 2 min; step 4) 50 degree C. for 5 min; step 5) increase to 60 degree C. at 0.1 degree C. per second; step 6) 55 degree C. for 2 min; step 7) 60 degree C. for 2 min; step 8) return to step 6 and repeat for 10 additional cycles. Incorporation of radioactivity permitted estimation of the yield of cDNA (Carninci and Hayashizaki, Methods Enzymol. 1999;303:19-44). The cDNA obtained was treated with proteinase K, extracted with phenol/chloroform and chloroform, and ethanol-precipitated using 5M NaCl.

The same procedure carried out for solution A was performed for solution B and cDNA obtained and treated in the same way.

RNA Removal.

Pellet was dissolved with 100 μl of H₂O and treated with the same volume of 150 mM NaOH/15 mM EDTA. After incubation at 45 degree C. for 10 min, following solutions were added: 100 μl of 1M Tris-HCl pH7.0 (we can combine two samples on this step), 2 μl RnaseI (10 U), 2 μl RNaseH (120u) (TAKARA) and incubated 37 degree C., 15 min. Again sample was treated with proteinase K, extracted with phenol/chloroform and chloroform, and ethanol-precipitated using 5M NaCl. Pellet dissolved in 100 μl of water was applied to S400 column. During this step it is possible to use the same column for the samples with the same direction. Sample was precipitated with Isopropanol and washed twice with 80% of ethanol.

Part 2. Hybridization and ExoVII—Restriction Enzyme Treatment.

Hybridization was carried out at Cot values of 1 to 20 in a buffer containing 40 percent formamide (from a deionized stock), 0.375M NaCl, 25 mM HEPES (pH 7.5), and 2.5 mM EDTA. Hybridization was carried out at 42 degree C. in a dry oven for 14 hrs. After hybridization, the sample was precipitated by adding 2.5 volumes of absolute ethanol and incubated for 30 minutes on ice. The sample was then centrifuged for 10 min at 15,000 rpm and washed twice with 70% ethanol; the hybrids were resuspended in 90 μl of water on ice.

Exonuclease VII treatment: for degradation of un-hybridized single stranded DNA was performed by addition of 10×L buffer (TAKARA) and 0.5 g 1 of enzyme. Reaction mix was incubation at 37 degree C. for 40 min. Later remained hybrids were treated with proteinase K, extracted with phenol/chloroform and chloroform, and ethanol-precipitated using 5M NaCl. Sample was resolved in 85 μl of TE. 5 μl of sample has been used for S1 nuclease check. We added of 0.5 μl 10×S1 buffer (300 mM Na acetate pH 4.5, 150 mM NaCl, 0.05 mM ZnSO₄) (TAKARA) to the sample, took 211 from the buffer-sample mixture and put on DE81 paper (Whatman) and checked the radioactivity (standard method). After that we add 2 μl of enzyme S1 (30 u) and incubate at 37 C for 30 min, took 2 μl and put them on DE81 paper (Whatman), S1 sensitive rate was calculated (Carninci and Hayashizaki, Methods Enzymol. 1999;303:19-44). Restriction Enzyme Digestion was done with the addition to the reaction mix (sample 80 10×l buffer 10 μl, BSAμl) of 2 μl HapII and 1.5 μl HpyCH4IV. After incubation at 37 degree C. or 2 h, 2 μl 5M NaCl and 1.5 μl AciI was applied and incubation was continued for another 2 hrs. All three restriction enzymes generate the same CG 5′ overhangs that will be farther used for the linker ligation. The three restriction enzymes used here in the EXAMPLES were selected to provide the same cloning site at the end of the fragments to allow for their direct ligation to the same linker as exemplified in EXAMPLES. However, the invention is not limited to the use of these enzymes as any other 4 bp cutter or as any other combination of 4 bp cutters or as any other combination of one or more 4 bp cutters together with any other restriction enzyme can be applied. In case of the use of other restriction enzymes than those used in this EXAMPLES the cloning sites of the linkers have to be adapted or the eventually sticky ends derived from the cleavage of the DNA have to be converted into blunt ends. Such adaptations of the linkers or the conversion of single stranded overhangs can be performed by standard techniques known to a person trained to the state of the art of molecular biology. Digested cDNA hybrids were treated with proteinase K, extracted with phenol/chloroform and chloroform, and ethanol-precipitated using 5M NaCl.

Part 3. Capture-Release.

The next step has been done to capture un-hybridized alternatively spliced exon loops (also called unpaired regions) using biotinylated random N′25mer oligonucleotides (Invitrogen). First of MPG-streptoavidin magnetic beads (CPG Inc.) were pretreated: 500 ul of Magnetic beads, 5 ul of 20 ug/ul tRNA were incubated on ice with occasional mixing for about 3 min. Washed with 1×CTAB Buffer (0.2M NaCl, 1 mM CTAB (Hexadecyltrimethylammonium bromide, Sigma), 10 mM EDTA, 25 mM Tris-HCl pH7.5) 3 times and added 500 μl of 1×CTAB Buffer, 5 μl of 20 μg/μl tRNA. Capture-Release was performed with N′ 25mer random oligonucleotides (Sambrook et al. Molecular cloning Lab. Manual, CSHL press, 1989) 5 μl (5 μg) first incubated on 94 degree C. for 30 sec. It was put on ice and 5 μg cDNA (hybridized) were applied to the mixture on ice. Then, it was incubated at 37 degree C. for 3 min. room temperature and the same volume of 2×CTAB Buffer (0.4M NaCl 2 mM CTAB 20 mM EDTA) was added at room temperature and incubated at 45 degree C. for 20 min (incubation can also be carried out at 37 degrees C. for 20 min or at room temperature for 20 min). After incubation, the sample was mixed with tRNA(Sigma) treated magnetic beads, rotated at room temperature for 30 min and washed with 500 μl 3M TMA Buffer (Tetramethylammonium Cloride, Sigma)(3M TMA, 20 mM EDTA, 50 mM Tris-HCL pH 7.5) 4 or 5 times. The radioactivity of the labeled samples was measured before and after the procedure in order to estimates the yield. 50 ul of 0.25× solution containing 4M Guanidium Thiocyanate, 0.5% n-lauryl sarcosine, 25 mM Sodium Citrate pH7.0 100 mM beta-mercaptoethanol with 0.5% Biotin and incubated 37 degree C. for 10 min. Supernatant was recovered and radioactivity was measured again. Steps were repeated until 80% or more cDNA hybrid was recovered. Sample was precipitated with isopropanol and in order to remove free biotin purification for 2-3 times has been done using Sepadex G50 (Amersham Pharmacia). Here capture release step can be repeated at least once again.

Part 4. Linker Ligation, PCR and Cloning.

Y shaped linkers were designed with GC 3′ overhangs that could ligate to 5° C./G overhands generated after the treatment of DNA hybrids with HpaII, HpyCH41V and AciI. 40 ng/μl of ASEL9. The two strands of the Y-shaped linker were the following:

| | |
|---|---|
| Up-5′ AAAAAGCAGGCTCGAGTCGAGTCGACGAGAG | (SEQ ID NO:3) |
| AGGC; | |
| Down 3′ P-CGGCCTCTCTCGGATCCGAATTCACCC | (SEQ ID NO:4) |
| AGCTT. | |

2.51 μl linkers were ligated to the 5 μl (about 200 ng) of DNA and for the complete reaction following reagents were added: 10×T4 ligase Buffer 0.75 μl, T4 DNA Ligase (both NEB), 1 μl of H20 and incubated at 16 degree C. overnight. Proteinase K treatment, extraction with phenol/chloroform and chloroform, and ethanol-precipitation using 5M NaCl was performed after the ligation step. Sample was resolved in 8 ul of TE and applied on electrophoresis (2% NuSieve GTG agarose, TAKARA). Portions of 60-80 bp of the above gel (for linker removal) were cut out and purified by Gel extraction kit(QIAGEN), 60 μl of water was applied to the filter unit for the recovering of cDNA hybrids. PCR was performed to amplify each strand of the hybrid containing alternatively spliced exons. Reaction was performed using following conditions: 0.75 μl of 10 mM primer

| | |
|---|---|
| ASEL9-1 | |
| GTGTGTGCGGCCGCACAAGTTTGTACAAAAAAGCAGG | (SEQ ID NO:5) |
| CTCGAGTCGA | |
| 75 μl of 10 mM ASEL9-2 | |
| CTTCTTGCGGCCGCACCACTTTGTACAAGAAAGCTGG | (SEQ ID NO:6) |
| GTGAATTCGGATC | |

2 ml of 10×Extaq Buffer (TAKARA, Japan), 4 ul 2.5 mM dNTPs (TAKARA, Japan), 0.4 μl *dGTP, 5 μl of template in total volume of 20 μl. Reaction mix was placed on PCR cycler(GeneAMP 9700, Applied Biosystems) with following conditions: 95 degree C. of hotstart add ExTaq 0.3 μl (TAKARA, Japan), 95 degree C. 30 sec, 55 degree C. for 1 min, 72 degree C. for 2 min about 4 or 8 cycles for the preparation of double stranded DNA for cloning purpose only. In another embodiment of the invention the PCR reaction can be performed with 20 cycles, or in yet another embodiment with 30 to 40 cycles to obtain sufficient amount of the PCR product for the direct use of the PCR product in other application rather than the cloning only. Proteinase K digestion was conducted followed by extraction with phenol/chloroform and chloroform (Carninci and Hayashizaki, Methods Enzymol. 1999; 303:19-44), and sample was dissolved with 40 μl of TE.

Cloning.

Cloning part included vector preparation (digestion and fragment purification with QIAGEN kit, QIAGEN), restriction digestion of cDNA fragments with BamHI and SalI and cloning of fragments into the vector. Vector pFLC1 (Carninci et al., September 2001, Vol. 77, (1-2):79-90). was double digested with 1 μl of SalI and 1 μl BamHI using 10 μl 10×SalI buffer (all NEB) and 10 μl of 10×BSA in total 100 μl and incubated at 37 degree C. for 1 hr. After Proteinase K treatment, extraction with phenol/chloroform and chloroform, and ethanol-precipitation using 5M NaCl, linear fragment of the vector was resolved in 100 μl of and applied on electrophoresis (0.8% NuSieve). The DNA fragment were cut out from the gel and purified by Gel Extraction kit (QIAGEN). Vector was dissolved in 100 μl of water. Digestion of PCR product as also performed with 1 μl of SalI and 1 μl BamHI using 10 μl 10×SalI buffer (all NEB) and 10 μl of 10×BSA in total 100 μl and incubated at 37 degree C. for 1 hr. After Proteinase K treatment, extraction with phenol/chloroform and chloroform, and ethanol-precipitation using 5M NaCl, linear fragment of the vector was resolved in 100 μl of and applied on electrophoresis (0.8% NuSieve). The probable location of dimmer was cut out from the gel and purified by Gel extraction kit (QIAGEN). Vector was dissolved in 100 μl of water.

Then, sample and vector were mixed and precipitated with 99% ETOH. Pellet was washed once with 70% ETOH and dried. After that the pellet was resolved directly with T4 ligation mixture (TAKARA), which was incubated at 16 degree C. for 12 hrs and then 5 min. at 65 degree C. Later, ligation mixture was transformed by electrophoration into DH10B E. coli competent cells.

Clone Isolation and Sequence Analysis.

After the titer check, bacterial clones were collected with commercially available picking machines (Q-bot and Q-pix; Genetics, UK) and transferred to 384-microwell plates. Duplicate plates were used to prepare plasmid DNA. E. coli clones containing vector DNAs from each of the 384-well plates were divided and grown in four 96-deepwell plates. After overnight growth, plasmids were extracted either manually (Itoh et al. 1997, Nucleic Acids Res 25:1315-1316) or automatically (Itoh et al. 1999, Genome Res. 9:463-470). Quality of insert was checked by digestion of individual clones with PvuII and applying on 0.8% agarose gel electrophoresis. Sequences were typically run on a RISA sequencing unit (Shimadzu, JAPAN) or using the Perkin Elmer-Applied Biosystems ABI 377 in accordance with standard sequencing methodologies such as described by Shibata et. all. Genome Res. 2000 November; 10(11).

Sequencing Results.

The above experiment made possible to obtain totally 46,159 clones. Inserts from all clones were sequenced using sequence line method described by Shibata et. al. November;10(11). Genome research 2000. It resulted in insert identification and mapping to the mouse genome from as many as 37.150 clones. The rest of data were difficult to localize mostly because of the small size (>=95%>=100 bp). Later on, all 37,150 clones were organized in 6,052 groups (each group included at least 2 clones), upon their sequence origin and this was followed with identification of alternative exon variants divided in total 467 subgroups

### Example 2

PCR Amplification of Inserts.

The present example has been carried out in the same way as EXAMPLE 1, with the difference that PCR has been carried out using the following T3GW2 and T7GW1 PCR primers in the first part of lambda-FLCII instead of primers T3GW1 and T7GW2, respectively.

| | |
|---|---|
| Primer T3GW2: | |
| GAGAGAGAGAATTAACCTCACTAAGGGACCACTTTGT | (SEQ ID NO:7) |
| ACAAGAAAGC | |
| and | |
| T7GW1: | |
| GAGAGAGAGTAATACGACTCACTATGGGACAAGTTTG | (SEQ ID NO:8) |
| TACAAAAAAGC. | |

### Example 3

This example as been carried out like EXAMPLE 1 with the difference that Part 4. Cloning has been carried out as follows.

Ligation to ClaI (Takara, Japan) digested pBlueScriptII (Stratagene, US) with T4 ligase 16 degree C. overnight, EtOH precipitation, resolve 5 μl 1 ul use for electrophoration to DH10B, titer check insert quality check with PvuII, and sequencing. All the steps as above were carried out in the same way as Example 1.

### Example 4

A Full-length cDNA libraries that are used for the a comparative analysis of alternative splicing, such as melanocyte and melanoma, are arrayed on 384 well plate (Shibata et al, Genome Res. 2000 November;10(11):1757-71.) and clones are transferred to nylon membranes (Gress T M et al, Mamm Genome. 1992;3(11):609-19.). Information derived from the alternative splicing such oligonucleotides are used as hybridization probe as in Gress et al. Colonies that are positive for the signals are recovered and subjected to full-insert sequence (Okazaki et al, Nature. 2002 Dec. 5;420(6915):563-573.) to obtain full-length information and physical clones of alternatively spliced cDNA.

### Example 5

Full-length cDNA libraries that have been used for the a comparative analysis of alternative splicing, such as melanocyte and melanoma, were arrayed on 384 well plate (Shibata et al, Genome Res. 2000 November;10(11):1757-71.) and followed by sequencing of 5′ and/or 3′ ends. After grouping the cDNAs (Konno et al, Genome Res. 2001 February;11(2):281-9.) they were aligned to fully sequenced cDNA clones or genome (Okazaki et al, Nature. 2002 December; 420(6915):563-573). Genome sequence and of a full-length were aligned into transcriptional units as described (Okazaki et al, Nature. 2002 Dec. 5;420(6915):563-573.) together with the 5′ and/or 3′ end sequences of the full-length cDNA libraries for which detection of alternative splicing was desired. Then, the information obtained at examples 1-3, which consists of part of cDNAs, was used for alignment to the transcriptional units previously obtained. This mapping allowed us listing up the candidate full-length cDNA that correspond to alternative splicing fragments of cDNAs of examples 1-3.

After in silico identification of the candidate clones, the candidate cDNAs were picked-up and subjected to full-insert sequencing as described (Okazaki et al, Nature. 2002 Dec. 5;420(6915):563-573) and alternatively spliced full-length cDNAs were obtained for further functional studies.

### Example 6

Full-length cDNA libraries that have been used for the a comparative analysis of alternative splicing, such as melanocyte and melanoma, were converted into plasmid DNAs (Carninci et al, Genomics. 2001 September;77(1-2):79-90.) and then into single strand DNAs (Bonaldo et al., Genome Res. 1996 September;6(9):791-806). The genetic information was used to prepare biotinylated oligonucleotides (Invitrogen) corresponding to the alternatively spliced cDNA (as in examples 1-3). Subsequently, single strand cDNA and biotinylated were mixed and hybridized as described in the Gentrap kit (Invitrogen) following the instruction of manufacturer. Alternatively splicing full-length cDNA from the libraries of interest were then recovered and after palting on agarose (Sambrook et al), the colonies were picked, subjected to one pass sequencing (Shibata et al, Genome Res. 2000 November;10(11):1757-71) and then the clones were subjected to full insert sequencing (Okazaki et al, Nature. 2002 Dec. 5;420(6915):563-573), obtaining alternatively spliced full-length cDNA.

1. A method for identifying, analyzing and/or cloning nucleic acid isoforms comprising the steps of:
a) preparing at least two nucleic acid isoforms, complementary to each other;
b) hybridizing the at least two complementary nucleic acid isoforms and forming double strand RNA/RNA or DNA/DNA hybrids comprising unpaired regions;
c) recovering the RNA/RNA or DNA/DNA hybrids comprising unpaired regions from not hybridized nucleic acids and from nucleic acids not comprising unpaired regions
d) identifying, analyzing and/or cloning the recovered nucleic acid fragment comprising unpaired regions.

2. The method of claim 1, wherein the recovering of step c) is carried out by using at least one restriction enzyme which cuts free single strand nucleic acids but does not cut double strand nucleic acids and/or at least a restriction enzyme which cut double strand nucleic acids but does not cut unpaired regions.

3. The method of claim 2, wherein the restriction enzyme which cuts free single strand nucleic acids but does not cut double strand nucleic acids is Exo VII, Exonuclease I, Exonuclease T, Lambda Exonuclease, T7 Exonuclease.

4. The method of claim 2, wherein at least one restriction enzyme which cuts double strand nucleic acids but does not cut unpaired regions is used.

5. The method of claim 4, wherein two restriction enzymes are used.

6. The method of claim 4, wherein the restriction enzymes cut at recognition sites comprising of 4 nucleotides of double strand nucleic acids but do not cut unpaired regions.

7. The method of claim 6, wherein the restriction enzymes are selected from HapII, HypCH4IV, AciI. HhaI, MspI, AluI, BstUI, DpnII, HaeIII, MboI, NlaIII, RsaI, Sau3AI, Taq alpha I and Tsp 509I.

8. The method of claim 1, wherein hybrids of RNA/RNA or DNA/DNA comprising unpaired regions are recovered from hybrid nucleic acids not comprising unpaired regions by using single strand nucleic acid-binding molecule.

9. The method of claim 8, wherein the single strand nucleic acid-binding molecule is bound to a tag.

10. The method of claim 8, wherein the nucleic acid to be recovered/single strand nucleic binding molecule/tag complex is recovered by use of a matrix which binds the tag.

11. The method of claim 8, wherein the single strand nucleic acid-binding molecule is a single strand nucleic acid-binding protein, antibody, antigen, oligonucleotide, a chemical group or chemical substance.

12. The method of claim 11, wherein the oligonucleotide which binds the tag is a random oligonucleotide.

13. The method of claim 12, wherein the random oligonucleotide is 15-30 nucleotides.

14. The method of claim 13, wherein the random oligonucleotide is 25 nucleotides.

15. The method of claim 8, wherein the tag is biotin, digoxigenin, antibody, antigen, a protein or nucleic acid binding molecule and the matrix is avidin, streptavidin, digoxigenin-binding molecule, an antibody or its ligand and/or chemical matrix.

16. The method of claim 8, wherein the tag is digoxigenin the matrix is a digoxigenin-binding molecule

17. The method of claim 8, wherein the tag is biotin and the matrix is avidin or streptavidin.

18. The method of claim 8, wherein the single strand nucleic acid-binding molecule is covalently attached to the matrix.

19. The method of claim 8, wherein the matrix is associated to a solid matrix surface selected from the group consisting of metal beads, magnetic beads, inorganic polymer beads, organic polymer beads, glass beads and agarose beads.

20. The method of claim 1, wherein hybrids of RNA/RNA or DNA/DNA comprising unpaired regions are recovered from hybrid nucleic acids and released from the single strand nucleic acid-binding molecule.

21. The method of claim 1, wherein the recovered nucleic acids comprising unpaired regions are bound with Y-shaped oriented linkers comprising a sticky end.

22. The method of claim 21, wherein the Y-shaped oriented linker comprises a different marker sequence in each single strand arm.

23. The method of claim 21, wherein the Y-shaped linker comprises a sticky end which hybridized with the sticky end of the fragment comprising the unpaired region.

24. The method of claim 23, wherein the sticky end of the Y-shaped linker hybridizes to the sticky end of the fragment comprising the unpaired region cut by the at least one restriction enzymes of claim 4.

25. The method of claim 1, wherein the at least two nucleic acid isoforms are prepared from at least one nucleic acid library, biological sample, cell, tissue, organ or biopsy.

26. The method of claim 25, wherein the two nucleic acid isoforms are prepared from two or more different nucleic acid libraries, biological samples, cells, tissues, organs or biopsies.

27. The method of claim 25, wherein the at least one of the at least two nucleic acid libraries, biological samples, cells, tissues, organs or biopsies is from tumoral source, from treated cells, and/or from cells undergoing apoptosis.

28. The method of claim 1, wherein the nucleic acids comprising nucleic acids comprising unpaired regions as recovered at step a), b), c) and/or d) of claim 1, are stored as nucleic acid isoforms-enriched libraries, used for the analysis of isoforms, or clones and/or used for the detection of further isoforms.

29. The method of claim 28, wherein the obtained libraries are alternative splicing-enriched libraries.

30. The method of claim 1, wherein the recovered nucleic acids comprising unpaired regions are amplified and cloned.

31. The method of claim 1, wherein the unpaired regions correspond to portions of genes that are differentially spliced.

32. The method of claim 1, wherein the unpaired regions correspond to portions of related genes derived from different loci within the same genome.

33. The method of claim 1, wherein the unpaired regions correspond to portions of unrelated genes derived from the same locus within a genome.

34. The method of claim 1, wherein the unpaired regions correspond to portions of related genes derived from different genomes.

35. The method of claim 1, wherein the recovered and cloned nucleic acid comprise the whole sequence of an unpaired region.

36. The method of claim 35, wherein the unpaired region corresponds to an exon or intron.

37. The method of claim 1, wherein the at least two complementary nucleic acid isoforms are prepared from starting materials by using at least two different RNA and/or DNA polymerases wherein each of the polymerases recognizes a different promoter site.

38. The method of claim 37, wherein RNA transcripts are obtained from the starting materials by using RNA polymerases which recognize a different promoter site, and cDNAs are prepared from the RNA transcripts by using reverse transcriptase.

39. The method of claim 38, wherein the at least two RNA polymerases recognizing different promoter site are selected from the group consisting of T3 RNA polymerase, T7 RNA polymerase, SP6 RNA polymerase and K11 RNA polymerase.

40. The method of claim 1, wherein a DNA polymerase and strand specific primers are used.

41. The method of claim 1, wherein a DNA polymerase and strand specific primers are used for linear amplification.

42. The method of claim 40, wherein the DNA polymerase is Taq DNA Polymerase or DNA Polymerase I Large (Klenow) Fragment, Exonuclease Minus

43. The method of claim 1, wherein the in step c) the nucleic acid isoforms are recovered by using linkers or primers.

44. The method of claim 43, wherein the linker or primer recognizes specific sequence sites.

45. The method of claim 43, wherein the isoform nucleic acids are recovered by using a linker and DNA or RNA ligase.

46. The method of claim 45, wherein the ligase is T4 DNA ligase, E. coli DNA ligase, RNA ligase or T4 RNA ligase.

47. The method of claim 1, wherein vectors or primers are used to introduce recognition sites for the 4 bp cutters at least one restriction enzymes of claim 4 at the ends of the nucleic acid isoforms.

48. The method of claim 1, wherein the nucleic acid isoforms are prepared from fragmented genomic DNA, cDNA, full-length cDNA, mRNA and/or RNA.

49. The method of claim 1, wherein the isoforms are full-length cDNAs or a fragment thereof comprising the unpaired region.

50. The method of claim 49, wherein the isoform substantially comprises the unpaired region.

51. A cloning vector comprising the isoform obtained according to the method of claim 1.

52. A host cell comprising the vector of claim 51.

53. A method for the preparation of isoform polypeptides comprising preparing the culture host cell of claim 52.

54. A method for preparing an isoform polypeptide comprising the step of preparing a isoform nucleic acid according the method of claim 1 and preparing the corresponding isoform polypeptide by using free-cell in-vitro or in vivo system.

55. A method for the identification of isoform polypeptides using the information obtained according to the method of claim 1.

56. A method for the detection and/or isolation of nucleic acid isoforms comprising the steps of:
l) preparing at least one oligonucleotide probe comprising the whole or part of sequence of an unpaired region identified or cloned according to the method of claim 1; and
m) hybridizing the oligonucleotide probe to nucleic acids comprising nucleic acid isoforms;
n) isolating the nucleic acid isoforms.

57. The method of claim 56, wherein the oligonucleotide probe is used to isolate full-length nucleic acid isoform.

58. The method of claim 56, wherein the oligonucleotide probe comprise at least part of or the entire sequence of one exon or intron.

59. A method for the determination of sequence variation of isoforms of claim 1, comprising the full-length or partial sequencing of the isoform.

60. The method of claim 1, wherein the sequence information of the sequence isoforms is used for the design of sequencing primers.

61. The method of claim 1, wherein the obtained isoform sequencing data are aligned to the genome, to genomic sequencing data and/or to cDNA sequencing data to obtain genetic information.

62. The method of claim 61, wherein the information is on alternative splicing.

63. A method of preparing a nucleic acid probe comprising obtaining information according to the method of claim 1.

64. The nucleic acid probe of claim 63.

65. A method for the detection or diagnosis of a disease, disease condition, pathology, a physiological condition, for assessing toxicity, for assessing the therapeutic potential of a test compound or for assessing the responsiveness of a patient to a test or treatment comprising obtaining information according to the method of claim 1.

66. Method for recovering of full-length cDNAs from cDNA libraries, biological samples, cells, tissues, organs or biopsies, from tumoral source, from treated cells, and/or from cells undergoing apoptosis by using the information on alternative splicing of claim 62.

67. Method for recovering of full-length cDNAs according to the method of claim 1 from cDNA libraries, biological samples, cells, tissues, organs or biopsies is from tumoral source, from treated cells, and/or from cells undergoing apoptosis by using the information on alternative splicing.

68. A non-soluble supports for hybridization in situ prepared with isoforms obtained according to the method of claim 1 or a nucleic acid probe prepared from information according to the method of claim 1.

69. A non soluble support comprising at least an nucleic acid comprising an unpaired region prepared according to the method of claim 1, a nucleic acid complementary to the unpaired region, and/or a nucleic acid probe prepared from information according to the method of claim 1, fixed, applied and/or printed thereon.

70. The support of claim 68, wherein said support is a solid matrix.

71. The support of claim 68, wherein said support is a microarray.

72. A method for the identification and isolation of nucleic acid isoform comprising obtaining the support of claim 68.

73. A method for in situ hybridization comprising obtaining the support of claim 68.

74. A method for the detection and/or diagnosis of a disease, disease condition, pathology, a physiological condition, for assessing toxicity, for assessing the therapeutic potential of a test compound, for assessing the responsiveness of a patient to a test or treatment, for the detection of nucleic acids or for the detection of nucleic acid isoforms comprising obtaining the support of claim 68.

75. A method for detecting and/or isolating nucleic acids from a support, microarray, nucleic acid library, biological sample, cell, tissue, organ and/or biopsy comprising obtaining genetic information by the method of claim 1.

76. A computer program or software applied on a medium for the analysis of genetic information obtained according to the method of claim 1.

77. A computer program or software applied on a medium for the alignment of the nucleic acid isoforms sequences or information obtained according to the method of claim 1 to genomic and/or cDNA sequence information.

78. A computer program or software applied on a medium for the prediction, determination and/or analysis of functional domains of polyeptides that derive from nucleic acid isoforms sequence or information obtained according to the method of claim 1.


SEQUENCE LISTING (8 sequences)

SEQ ID NO: 1 - DNA, 49 bp, Artificial
  PCR Primer T3GW1
gagagagagaattaaccctcactaaagggacaagtttgtacaaaaaagc

SEQ ID NO: 2 - DNA, 47 bp, Artificial
  PCR primer T7GW2
gagagagagaattaacctcactaagggaccactttgtacaagaaagc

SEQ ID NO: 3 - DNA, 35 bp, Artificial
  Up strand of the Y-shaped linker
aaaaagcaggctcgagtcgagtcgacgagagaggc

SEQ ID NO: 4 - DNA, 32 bp, Artificial
  Down strand of the Y-shaped linker
cggcctctctcggatccgaattcacccagctt

SEQ ID NO: 5 - DNA, 47 bp, Artificial
  PCR primer ASEL9-1
gtgtgtgcggccgcacaagtttgtacaaaaaagcaggctcgagtcga

SEQ ID NO: 6 - DNA, 50 bp, Artificial
  PCR primer ASEL9-2
cttcttgcggccgcaccactttgtacaagaaagctgggtgaattcggatc

SEQ ID NO: 7 - DNA, 47 bp, Artificial; identical to SEQ ID NO: 2
  PCR Primer T3GW2

SEQ ID NO: 8 - DNA, 48 bp, Artificial
  PCR primer T7GW1
gagagagagtaatacgactcactatgggacaagtttgtacaaaaaagc